(19) United States
(12) Patent Application Publication
(10) Pub. No.: US 2009/0292245 A1
(43) Pub. Date: Nov. 26, 2009

(54) MICROPUMP-OPERATED DRUG DOSING SYSTEM

(75) Inventors: Nils BASSO, Aschaffenburg (DE); Christian POMMEREAU, Undenheim (DE); Alastair CLARKE, Nantwich (GB); Rene RICHTER, Tharandt (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt am Main (DE)

(21) Appl. No.: 12/415,222

(22) Filed: Mar. 31, 2009

Related U.S. Application Data
(63) Continuation of application No. PCT/EP2007/008362, filed on Sep. 26, 2007.

(30) Foreign Application Priority Data
Oct. 7, 2006 (DE) 102006047538.0

(51) Int. Cl. A61M 5/142; A61M 1/00

(52) U.S. Cl. 604/122; 604/151

(57) ABSTRACT

The present invention relates to a device for injecting a substance into the human or animal body, where the medicament to be injected is removed from a reservoir by generating a subatmospheric pressure.

The invention relates to a device for injecting a substance into the human or animal body, where the medicament to be injected is removed from a reservoir by generating a subatmospheric pressure.

Many pharmaceuticals must be injected into the body. This applies in particular to those which are inactive or are crucially low of activity on oral administration. These pharmaceuticals include in particular proteins (such as, for example, insulin, growth hormones, interferons), carbohydrates (e.g. heparin), antibodies or most vaccines. Syringes, medicament pens or medicament pumps are predominantly used for injection into the body.

The conventional insulin injection apparatus is the insulin syringe. This has been used since the start of insulin therapy, but has in recent years been displaced stepwise by introduction of the insulin pen, especially in Germany. Nevertheless, syringes are at present irreplaceable, e.g. if an insulin pen is lost or defective, and are used by many diabetics in combination with insulin pens. The freedom from maintenance and the universal availability is advantageous, especially during journeys.

Insulin syringes differ in their designation and graduation according to the concentration of the insulin to be used, U40 or U100. The insulin can be taken either from vials or else from the prefilled cartridges for insulin pens. This makes it possible to mix different types of insulin and reduces the number of injections necessary. Particular care about freedom from bubbles is necessary when the insulin is drawn into the syringe. The directly visible insulin dose which has been drawn in makes it possible for the user easily to check the amount of insulin injected. Nevertheless, skill and regular use are necessary for error-free administration with insulin syringes.

A further injection apparatus which is now very widely used around the world and especially in Europe is the insulin pen.

This medical apparatus which is the size of a marker pen was developed in the mid 1980's and is employed mainly for more intensive insulin therapy. A substantial innovation compared with insulin syringes is the use of an exchangeable medicament container. This container, also called, carpule or cartridge, is filled with insulin when supplied by the manufacturer and is inserted into the insulin pen before use. When the pen is operated, a needle pierces the sealing disk of the cartridge and achieves parenteral injection of the preselected dose on administration of the insulin. An injection and release mechanism generates during the injection an injection stroke which advances a plunger or stopper in the cartridge and causes the preselected dose to be delivered into the target tissue. The mechanism usually consists of a rigid plunger stem with an overall length corresponding to the cartridge stopper stroke.

Insulin pens are divided into disposable and reusable ones. In the case of disposable ones, the cartridge and the metering mechanism form a unit prefabricated by the manufacturer and are disposed of together after the cartridge is emptied. Reuse of the metering mechanism is not intended. In contrast to prefabricated pens, reusable pens make increased demands on the user. Thus, when the cartridge is changed, the plunger stem must be retracted into the starting position. This takes place, depending on the model, by twisting or sliding the plunger stem while simultaneously actuating a special function in the metering mechanism. This must be carried out very carefully by the user because malfunctions, e.g. sticking of the plunger stem, may occur occasionally owing to the daily use and the high mechanical stress.

Reusable insulin pens are further divided into manual and semiautomatic pens. In the case of manual pens, the user exerts a force with the finger to actuate the injection button and thus determines the duration and progress of the injection. By contrast, with semiautomatic insulin pens, use is preceded by a manual tensioning of a spring which stores the necessary energy for injection. In the actual injection step, the spring is released by the user. The speed of injection is fixed by the power of the spring and cannot be adapted to personal needs.

EP 1045146 discloses a medical metering pump in which a pump is attached between a container for a liquid medicament to be administered and a removal line. The system is used to operate a continuously operating medical metering pump which is affixed to the patient's body during operation.

A medical metering pump is configured for delivering a medicament in soluble form continuously over a prolonged period (for example from about 10 minutes up to several hours). A medical metering pump is to be differentiated herein in particular from an injection device such as, for example, a syringe or a medicament pen such as, for example, an insulin pen. With a medicament pen, a previously fixed amount of a medicament is delivered within a short time in the region of, for example, less than 1 second, 1 to 30 seconds, 1 to 60 seconds up to 1 to 2 minutes.

Continuous medicament delivery by means of a medical metering pump has the disadvantage that medical problems may occur at the site of input, e.g. through rejection reactions of the body, contamination of the material or injuries by the cannula, over the relatively long time of input. Continuous medicament input requires a different treatment regimen for a disease than noncontinuous input of the medicament. Insulin pumps are generally employed for type 1 diabetics.

WO 2006/003130 describes an injection device by means of a suction device in which the suction pump is operated by a mechanical spring force. The mechanical drive is in this case not amenable to control, monitoring and signal processing by means of electronic components and software. The result thereof is that a pharmaceutical can be administered only in the mechanical context. The use of a mechanical spring moreover leads to a jerkily quick and thus painful injection result.

In the injection device of FR2321903,the pharmaceutical is removed from the reservoir by subatmospheric pressure. The subatmospheric pressure is generated by mechanical movements of parts of the apparatus toward one another. This apparatus is also subject to the restricted range of application of a purely mechanical system. In addition, people with reduced mobility or muscle power will be able to use this system only very restrictedly or not at all.

The device according to the invention can therefore be employed very much more flexibly by comparison with the known prior art, crucially facilitates the injection process for the patient, can be adapted in a simple manner to receive reservoirs of entirely different design (also from different manufacturers), is amenable to control and monitoring by electronic systems, is suitable for data acquisition and data exchange and can be operated under remote control.

The invention thus relates to a device for injecting a substance into the human or animal body, comprising inter alia
a) at least one reservoir; and
b) one or more outflow lines from a reservoir from a); and
c) a pump mechanism which is attached between a reservoir from a) and an outflow line from b); and
d) a component which is suitable for injection and which is functionally connected to an outflow line from b);
   wherein the pump mechanism is driven by motive power.

A device consists of one or more components and serves a particular medical purpose, in particular injection of a substance into the human or animal body. One component consists of one or more elements and serves to comply with a technical or non-technical function. A function is technical if it relates to a transfer of force, work, energy, material (substance), data and/or signals, the maintenance of the structure and/or form or the storage of a substance, or storage of information. A function is not technical if it relates to the input or output of information by or to the user of the device or of a substance by or to the user of the device.

A component may be for example part of a technical apparatus which provides a partial function in relation to the overall function of the apparatus. A component is for example a reservoir. Reservoir may be an exchangeable cartridge comprising a substance (in particular a medicament such as, for example, insulin). The exchangeable cartridge may be suitable in particular for use in an insulin pen or another device for injecting a medicament into the human or animal body. Another example of a technical component is a device for pumping or a pump. Further examples of technical components are in particular syringes, needles, plunger stems, metering units, mechanical displays, tubing, seals, batteries, motors, transmissions, electronic displays, electronic memories or electronic controls. A purpose in connection with the technical device is intended to be in particular the movement of liquid from one place to another. One purpose is for example defined by moving a liquid volume from a reservoir to an outflow line. The purpose may also be injection of a medicament into the human or animal body.

A component may be connected in a technical manner to one or more other components in order to comply with a purpose together. A technical connection is for example a connection of components which is suitable for transmitting force, work, energy, material (substance), data and/or signals. The components can be connected for example via a mechanical coupling, a fixed mechanical connection (gluing, screwing, riveting, via linkage or the like), a toothed wheel, a latch, an interlock means, a metallic wire, an optical waveguide, a radio link, an electromagnetic field, a light beam or the like.

A reservoir is distinguished by external shape and an internal volume present therein and in which a substance, in particular a liquid, is enclosed. The volume is closed fluid-tight to the outside. However, access routes into the volume are present and allow input and/or removal of the substance. The external shape can be produced by processing glass, metal (e.g. aluminum) or plastics. Access may be through a perforatable membrane or a screw closure. A reservoir is for example an insulin cartridge for use in an insulin pen.

Injection is the introduction of substances in particular of liquids by means of a cannula together with syringe or functionally comparable device such as in particular a pen into the human or animal body. Inter alia, subcutaneous, intramuscular, intravenous, intracutaneous and intraarticular injection are known. Subcutaneous injection takes place underneath the skin and is relatively easy to carry out, not very painful and can be undertaken by the patient himself. Intramuscular injection takes place into a muscle. Since greater risks exist in this case, such as, for example, painful periosteal injury, this is usually undertaken by medical staff. Intravenous injection takes place following venepuncture directly through a vein.

In intracutaneous injection, a pharmaceutical is passed directly under the dermis. In intraarticular injection, a liquid is injected into a joint. Injection of a substance into the human or animal body is to be distinguished in particular from introduction of a substance through a medicament pump, an infusion or another type of continuous supply taking place over a certain time.

A pump mechanism is a functional unit consisting of one or more technical components for moving liquids. The pump mechanism in the sense of the present invention may be composed of at least one pumping component and of at least one further component which supplies the pumping component with drive energy or may consist thereof. A pumping component is for example a tubing pump, diaphragm pump, gear pump or a piezoelectrically operated pump. A further component which supplies the pumping component with drive energy may be for example an electric motor.

A pump mechanism in the sense of this invention may include at least one pumping component and in addition interfaces to this pumping component, via which an externally present technical apparatus for generating motive power can be connected to the pumping component or via which a technical apparatus for generating motive power can be coupled to the pumping component. An interface relates in this connection in particular to the mechanical connection of the drive shaft of a technical apparatus for generating motive power to the apparatus part, which generates the pumping action, of the pumping component, such as, for example, a pump driven by an electric motor. Such an interface also includes mechanical holders and possibly required electrical contacts or contacts to transfer information, data and/or signals. An apparatus such as, for example, an electric motor is externally present if it is not a constituent of the device from the outset but is provided subsequently in order to be held together with the device in a functional manner via its own interfaces attached to the technical device therefor. A technical apparatus for generating motive power such as, for example, an electric motor is functionally connected to the pumping component, for example a tubing pump, if technical apparatus and component can be distinguished as respectively separate apparatus units, for example by a spatial distance between the apparatuses. This does not stand in the way of functional connection, which can be maintained for example by tubes, wires, long-distance couplings and the like.

A technical apparatus for generating motive power, such as, for example, an electric motor is coupled to the pumping component, for example a tubing pump, if the two apparatuses appear, after connection via the interfaces, as a single apparatus, for example can be moved only simultaneously and only together as assembled unit.

The connection of the technical apparatus to generate the motive power (for example an electric motor) is functionally connected to the pumping component (for example a tubing pump) when the drive movement of the shaft of such an apparatus is converted by suitable technical connecting members of the pumping component into a pumping action of the pumping component. Suitable technical connecting members for such a functional connection are for example fixed linkages or releasable couplings between the shaft of the driving technical apparatus and the shaft of the pumping component.

The pump mechanism in the sense of this invention may in a preferred embodiment consist of a pumping component which is present together with a component providing the drive energy in completely integrated form, such as, for example, in the form of a motor-driven pump.

The pumping component of an invention as described above can, in preferred drive forms, consist of a tubing pump, a diaphragm pump or a piezoelectrically operated pump. However, it is also possible in addition to use bellows pumps, piston pumps, rotary piston pumps, gear pumps, rotating disk pumps, belt pumps, eccentric screw pumps, propeller pumps and others.

A pumping component such as, for example, a pump is a machine by means of which the energy present in a liquid is increased by furnishing mechanical work. Either the pressure of the liquid is increased, or kinetic energy is also given to the liquid. It is thus possible, when suitable technical equipment is available, to achieve a directed translocation of the liquid. Mechanical work can be furnished by machines designed for this purpose, such as, for example, electric motors. An electric motor can convert electrical or chemical work with the aid of magnetic fields into mechanical work. Electric motors can be operated with direct current, three-phase current or alternating current. In a preferred embodiment of the invention as described above, the motive power for driving the pumping component is generated by an electric motor. Such a motive power can, however, also be furnished for example by a solar cell motor, a gas engine, a motor operated by vapor pressure, a motor operated by transforming mechanical energy, or the like.

The energy source preferably used to operate the component which supplies the motive power, in particular an electric motor, is a battery, an accumulator and/or a solar cell, and/or domestic current (where appropriate via a transformer).

A releasable and reconnectable coupling and/or a transmission for stepping down, stepping up, synchronization or for transforming a type of motion is inserted between pumping component and the component for supplying the pumping component with motive power.

A transmission is intended to mean a mechanical component by means of which a rotary motion can be transmitted or transformed. Transformation means for example conversion of a rotary motion into a horizontal or vertical reciprocating motion. Stepping up, stepping down and synchronization means a corresponding ratio of the input to output speeds or torques.

In a preferred embodiment of the invention in the form of the technical device as described above, the reservoir has an inflexible outer wall. This wall can consist for example of glass, of metal, in particular steel, aluminum, titanium, gold, silver, platinum, of wood, of plastic, in particular a polycarbonate or acrylic glass, a composite material composed of one or more of the previously mentioned substances, or another material. A reservoir in the sense of this invention is in particular a bottle or cartridge in which a pharmaceutical is stored or can be stored. Reservoirs of these types are obtainable for example as insulin cartridges for use in insulin pens or insulin pumps from various manufacturers (e.g. Sanofi-Aventis; Novo Nordisk; Eli Lilly) in the pharmaceutical trade, especially in pharmacies.

In another preferred embodiment of the invention, the reservoir has a flexible outer wall. Liquid can be removed from such a reservoir with flexible wall for example by generating a subatmospheric pressure and thus causing the reservoir to be squeezed by the action of the external air pressure or the pressure in a pressurized chamber.

In a further preferred embodiment, the reservoir consists of a commercially available vessel and/or cartridge in each case containing or suitable for storing a medicament. Such a medicament is preferably insulin.

A component preferably used as outflow line from a reservoir is one having a cavity. This component is aligned with one end toward the reservoir and is connected thereto, and is aligned with another end toward the pump mechanism and is connected thereto. The connection can be produced by conventional connection techniques for workpieces such as, for example, gluing, welding, riveting, screwing, clamping on, flanging on and other technicians. The outflow line may also be formed from part of the reservoir by inserting a cavity into such a part and producing an accurately fitting connection to the pump mechanism or any adapter necessary. The accurately fitting connection can be effected by appropriate external shaping of the reservoir.

The outflow line can likewise be formed from part of the pump mechanism, by inserting a cavity into such a party and producing an accurately fitting connection to the reservoir or any adapter necessary. The accurately fitting connection can be effected by appropriate external shaping of the pump mechanism. It is also possible to use as outflow line for example a tubular structure or a tubing made of metal, in particular steel, aluminum or of plastic or of another material.

The outflow line has an internal cavity which is suitable for removing a liquid from the reservoir. The cavity usually has a cylindrical shape. The connection of the outflow line to the reservoir, to the pump mechanism and any other components is designed to be fluid-tight as far as possible. The outflow line is functional when the substance, in particular a liquid, can be removed from the reservoir therewith.

A component for injecting a substance as component of the device according to the invention consists in a preferred embodiment in particular of a cannula.

A cannula is essentially a hollow needle which is usually made of metal (e.g. steel, stainless steel, gold, silver, platinum). The end of the cannula is frequently sharpened by grinding at an angle. The cannula may be pointed and/or sharpened at one end and blunt at the other end, but it may also be pointed and/or sharpened at both ends. The cannula has at one of the two ends a usually conical attachment made of, for example, plastic by means of which the hollow needle can be arranged for example by pushing or screwing onto a medical apparatus such as, for example, a syringe, a medicament pen, in particular an insulin pen, a medicament container or a medicament pump. The cannula serves in functional interaction with a syringe, a pen, a pump or another medical apparatus suitable for the purpose, to remove or supply a liquid from or into the human or animal body.

The diameter of the cannula (external diameter) is usually stated in mm or in gauge (18 gauge=1.2 mm; 20 gauge=0.9 mm; 21 gauge=0.8 mm; 22 gauge=0.7 mm; 23 gauge=0.6 mm; 25 gauge=0.5 mm; 27 gauge=0.4 mm). Another parameter for characterizing the cannula is its length. Typical lengths of cannulas are 40 mm, 30 mm, 25 mm, 8 mm, 6 mm and other lengths.

In a preferred embodiment of the invention, the technical device includes at least one electronic component for checking, monitoring and/or controlling the pumping component and/or the component which supplies the pumping component with motive power. In a further preferred embodiment of the invention, the technical device includes a flow sensor to determine the amount of the substance which is removed from the reservoir and/or the amount used for the injection.

The invention further relates to the production of a device as described above, where
a) a component to receive a reservoir is provided;
b) a reservoir is provided. (This reservoir may comprise a pharmaceutical in liquid form, for example insulin. The reservoir may, however, also be present in empty form);
c) an outflow line from the reservoir is provided;
d) a pump mechanism is provided;
e) a component for injecting a substance is provided;
f) possibly a flow sensor is provided;
g) possibly electronic components for storage and/or data processing and/or data transfer are provided;
h) the individual constituents as described in a) to g) are joined together to give a functional unit.

A technical device according to the invention is suitable for example as constituent of an apparatus which is suitable for injecting a substance into the human or animal body avoiding the gastrointestinal tract. It is possible with such an apparatus to administer preferably pharmaceuticals and in particular insulin.

The present invention further relates to a medical apparatus for injecting a pharmaceutical into the human or animal body, comprising inter alia the following constituents a) to f) or consisting wholly or partly of the following constituents a) to f):
a) a base element for mounting at least one further component;
b) a component for removing air bubbles from the liquid intended for injection;
c) a component for presetting the amount of liquid intended for injection;
d) a component for displaying the amount of liquid intended for injection;
e) a component for initiating the injection of liquid;
f) a component consisting of one or more of the technical devices according to the invention described above.

This medical apparatus includes in a preferred embodiment at least one means for storing and/or processing data and/or signals.

This medical apparatus includes in a further preferred embodiment in addition an interface for transmitting data and/or signals to and/or from an external technical unit which is appropriately configured for the storage and/or processing of data and/or signals. Such an external technical unit may consist for example of a PC together with software installed thereon for the storage and/or processing of data and/or signals which are transmitted by a medical apparatus.

Such a medical apparatus comprises in a preferred embodiment insulin, in particular a long-acting and/or a short-acting insulin and can accordingly be employed to inject an insulin, in particular a long-acting and/or a short-acting insulin.

Such a medical apparatus comprises in another preferred embodiment GLP-1 and can accordingly be employed for injecting GLP-1. Such a medical apparatus comprises in a further preferred embodiment Lovenox and can accordingly be used to inject Lovenox.

The medical apparatus according to the invention comprises a pharmaceutical such as, in particular, insulin, for example in long-acting or short-acting form, GLP-1 or Lovenox in a reservoir. Said pharmaceuticals, and all other pharmaceuticals which can be injected by means of the apparatus according to the invention, are in this connection present in solution or, depending on the solubility behavior of the substance under different temperature or pressure conditions (for example storage conditions), as suspension or partly in liquid and partly in solid form. The pharmaceutical for injection by means of the medical apparatus according to the invention may also be provided in a reservoir having two or more separate chambers, where one chamber comprises the pharmaceutical in solid form, and a further chamber comprises a liquid such as, for example, water with or without additions such as buffers, ions, preservatives, stabilizers, acids, bases, alcohols, organic solvents inter alia. Before injection of the pharmaceutical, it is converted into the soluble form. This takes place for example by a device which makes the separation of the chambers between the pharmaceutical in solid form and the liquid (for example water) permeable so that the liquid (for example water) can come into contact with the pharmaceutical. The pharmaceutical can be converted into the soluble form by further measures such as shaking, stirring, reciprocal motion or the like by the same device which makes the separation between the chambers permeable, or another device which is suitable therefor, or by manual actuation of the user, before it is then injected.

The invention relates in a further embodiment to the production of a medical apparatus according to the invention, where
a) a base element for mounting at least one further component is provided;
b) a component for removing air bubbles from the liquid intended for injection is provided;
c) a component for presetting the amount of the liquid intended for injection is provided;
d) a component in the form of a display is provided;
e) a component in the form of a release mechanism is provided;
f) at least one technical device according to the invention as described above is provided;
g) the individual constituents from a) to f) are assembled to give a functional unit.

The invention further relates to the use of a medical apparatus according to the invention for the prophylaxis and/or therapy of a disease and/or dysfunction of the body by means of a substance whose pharmacological activity is diminished or lost in the gastrointestinal tract. Such a substance is for example a protein, carbohydrate, a nucleic acid or a vaccine. Examples of such substances are insulins, growth hormones, interferons, interleukins, cytokines, heparins, monoclonal antibodies, attenuated pathogens of viral infections (e.g. influenza) and others.

The use of a medical apparatus according to the invention relates inter alia to the treatment of diabetes, the administration of insulin, GLP-1, an interferon, growth hormone, heparin, Lovenox or a vaccine.

A medical apparatus in the sense of this invention is used for the therapy of the human or animal body in particular by supplying a substance such as, for example, insulin to the human or animal body.

Supplying a substance can take place by injection such as, for example, by a syringe or a medicament pen, in particular an insulin pen. Supply by an insulin pump differs from injection by taking place in a continuous manner and is to be distinguished from an injection in the sense of this invention.

A medical apparatus is in particular an apparatus for injecting the substance into the human or animal body. Besides the syringe, it is possible for such an apparatus for injection to be a medicament pen such as, for example, an insulin pen. Medicament pens are suitable in various form and for various purposes and are obtainable on the market from various manufacturers (e.g. Optiklick, Optipen, Optiset).

The base element of a medical apparatus such as, for example, an insulin pen is intended to mean its outer casing, which also decisively determines the shape. This shape may be for example elongate similar to a pen, oval, round, square, rectangular, in the shape of an egg timer, hingedly openable or telescopically collapsible. The material of the outer casing may be made from one or more plastics, from glass, metal, wood or ceramic. The mounting of a component in or on a base element means that this component is present in the base element or attached to the base element in the resulting medical apparatus ready for use.

Every insulin pen must satisfy numerous requirements in relation to ease of operation in order to make safe and fault-free use possible. The basic requirement is for display of the preselected dose and of the amount remaining in the cartridge. The setting of the dose, and completion of the injection process should moreover be made audible, perceptible by touch and visible. This safety requirement arises in particular from the limited perception capacities of elderly type 2 diabetes patients.

Besides insulin pens with needles, also employed for insulin therapy are needle-free injection systems. A current example of the use of needle-free injection systems is the Injex injection system of Rösch AG. With this injector, extremely high pressure is used to shoot the insulin through a microneedle into the adipose layer of the skin. An elastic spring which is tensioned manually before injection stores the necessary injection energy therefor. The injected material is in this case distributed homogeneously and conically in the adipose tissue.

A non-negligible advantage of this apparatus is the needle-free injection of the medicament, which in some patients reduces the psychological inhibition threshold for insulin administration. In addition, needle-free injection precludes infection of the puncture site. Disadvantages compared with conventional insulin pens proved to be the transfer of the insulin into special cartridges, the comparatively larger mass of the apparatus, and the inclusion of further accessories for tensioning the spring.

Insulin pumps differ from insulin syringes by being completely automatic infusion systems for continuous subcutaneous injection of insulin. They have approximately the size of a cigarette pack and are worn permanently on the body. Short-acting insulin is injected through a catheter and a needle located in the skin into the cutaneous tissue according to the program preset by the patient. The task of the insulin pump is to imitate the continuous output of insulin by the pancreas to reduce the blood glucose level, but without being able to regulate the blood glucose with closed-loop control. Because of the continuous and adaptable supply of insulin, these pumps have advantages in particular for people engaged in sporting activities and whose daily routine varies greatly. It is possible with insulin pump therapy to compensate for large variations in blood glucose, e.g. in diabetics with a pronounced DAWN phenomenon, which can be controlled with conventional methods only with increased effort. One disadvantage is that when the insulin supply is interrupted owing to the lack of an insulin reservoir in the human body, severe metabolic derangement may occur. Insulin pumps are available in various technical configurations, and apparatuses with syringe-like containers have become established during the technical development. In analogy to the insulin pens with needles, the insulin is present in a reservoir with moveable stopper. The latter is moved by a motor-driven plunger stem.

Owing to the completely automatic and continuous delivery of insulin, the pumps are provided with a large number of security systems in order to protect the user from malfunctions with serious consequences. However, this does not mean that responsible and anticipatory use of the apparatus is unnecessary.

On the basis of the current injection apparatuses and further technological development in medical and microsystems technology there is an evident trend to completely automatic miniaturized medicament metering systems. Further development might go in the direction of implantable and extracorporeal medicament metering systems. The aim of implantable insulin pumps is to free the diabetic from the daily injection of insulin without the need to wear an external apparatus on the body.

Insulin pens are concentrate in the essential ergonomic and safety features in the EN ISO standard 11608. This likewise includes the geometric/material properties of the insulin cartridges and pen needles. The handling and the operation of a pen is thus substantially uniform and independent of the model for the user.

The contents of the EN ISO standard 11608 where this relates to insulin pens, insulin cartridges and needles is hereby expressly incorporated in the present disclosure by reference.

In the design of the pens there are some considerable differences to be found in the pens of the various manufacturers. The reasons therefor are for example the designation for different target groups (children, elderly people). Because of the requirements of the EN ISO standard 11608, the differences are confined in particular to the injection mechanism and the release mechanism. The dose selector and the dose display are mostly subject to ergonomic requirements and result from the general design conditions of the respective model.

The essential functional element of an insulin pen is the injection mechanism. It determines the type and size of the pen and the design of the release mechanism and of the dose selector. The mechanism translates the dose preset on the dose selector with the injection energy derived from the release mechanism into an injection stroke of the stopper in the cartridge. This energy is transmitted either directly to the injection mechanism or through a motion-modifying transmission.

It is technically possible for the injection mechanism in the shape of the plunger stem to vary in form.

In the insulin pens currently available on the market, solutions with a rigid (e.g. threaded spindle, toothed rack) or a flexible (e.g. curved toothed rack, curved compression spring) design have become established. Other possible configurations such as telescopic plunger stem (e.g. screw mechanism, belt and chain drive, hydraulic transmission, coupled transmission) are not employed in the insulin pens currently commercially available.

The design solutions of the rigid and flexible type vary widely and depend on the kind of pen, i.e. reusable pen or disposable pen. Plunger stems employed are threaded spindles or toothed racks or combinations of the two. In the dose selector, an angle of rotation corresponding to the dose is preset with the aid of detent devices and is transmitted by subsequent screw mechanisms and toothed gears to the injection mechanism and transformed into the injection stroke.

Delivery of the medicament takes place by specifying an injection stroke and the resulting displacement of the stopper. The amount of liquid delivered depends on the injection stroke and the internal diameter of the cartridge. To avoid dosage errors, air bubbles must be completely removed in accordance with manufacturers' specifications and the EN ISO standard 11608. In addition, after delivery of the liquid, a sufficiently long time should be allowed to elapse in order to ensure a steady state, i.e. normal pressure of the liquid and relaxation of the stopper in the cartridge.

The reservoir for the medicament (also referred to as cartridge) influences the construction and functional structure of the medicament pen. Partial functions which can be distinguished in this connection are firstly a protective function for the medicament, then a conveying function and finally a coupling function to the injection system of the medicament pen. The protective function is achieved by the cartridge as a whole, i.e. by stopper, glass body and sealing disk. The conveying function for the medicament is conferred by the stopper, which is displaced with the aid of the injection mechanism and brings about a change in volume in the cartridge. The coupling function to the injection system finally is produced by sealing means (e.g. sealing disk).

In an automatic medicament pen (e.g. automatic insulin pen), the injection energy is applied by a drive with subsequent transmission. An energy supply and control unit are additionally necessary.

In the injection mechanism according to the invention, the medicament (e.g. through insulin) is conveyed not by displacing the stopper by means of an injection mechanism, but by introducing a pump device. The pump device is inserted between cartridge and injection system and is to be provided with appropriate interfaces.

The pump device can be provided with a flow sensor. It is in direct contact with the medicament, e.g. insulin, thus giving rise to additional requirements such as reduced organism count, sterility, biscompatibility inter alia. On application of this functional principle, numerous variables (e.g. the liquid pressure in the medicament container) are altered by comparison with a conventional medicament pen for injection (e.g. an insulin pen), because a subatmospheric pressure arises when the medicament is sucked out.

Insulin cartridges serve as primary packaging for the medicament and must satisfy high standards. This relates to the dimensional accuracy of the cartridge in relation to the accuracy of dosage and compatibility with other components. The EN ISO standard 11608-3 is concerned with these requirements and describes the fundamental aspects and the geometrical/material construction without unnecessarily restricting the shape of the cartridge. The pharmaceutical impermeability of the cartridge must likewise be ensured.

The cartridges consist of a plurality of subcomponents. The principal one is the cylinder of pharmaceutical glass with high neutrality and chemical resistance to insulin. Before filling, the surface quality of the cylinder is improved by siliconization. This surface treatment reduces the sliding and breakaway forces of the stopper, increases the accuracy of dosage and reduces the dissolving out of glass constituents during a long storage time. The degree of siliconization correlates in this connection with the level of the frictional forces of the stopper, a limit being set by the sensitivity of the insulin to the silicone.

The cartridge is sealed at both ends by elastomeric closure parts, the stopper and the sealing disk. Crucial points in this connection are the demonstrated mechanical impermeability in various pressure situations, and the microbiological impermeability to organisms in long-term tests. Further important points are the maximum allowable stopper forces and the number of punctures of the sealing disk with a cannula.

Pen needles are sterile disposable products employed to guide the insulin out of the cartridge into the target tissue. They are subject, just like cartridges, to strict requirements because the real functionality of the insulin pen is achieved only through cooperation of the two components. The needle consists of a cannula which is ground at both ends and which is set in a cartridge attachment piece. Optimized grinding of cannulas makes it possible for insertion into the target tissue to be substantially painless for the patient and causes only slight tissue damage on withdrawal again. Likewise, the cartridge sealing disk is pierced without extensive fragmentation. This is an obligatory requirement because the impermeability of the cartridge must be ensured also when the needle is regularly changed. The cartridge attachment piece ensures a firm fit on the insulin pen.

Even if pen needles show signs of wear which are scarcely visible to the eye after being used two or more times, they should nevertheless be changed after each injection for reasons of sterility. In addition, crystallized insulin may block the needle. Moreover, air gets into the cartridge if there are temperature variations, which equally causes dosage errors. Thus, a temperature change of only 15 K causes up to 15 μl of air to enter the cartridge.

Microfluidics is a subsection of microsystems technology and includes the design, production, use and investigation of microsystems which manipulate and treat amounts of fluid in channel cross sections with dimensions of from 1 μm to 1 mm. Microfluidic systems are employed in medical technology, biochemistry, chemical engineering and analysis, and microreaction technology. These microsystems may have dimensions in the millimeter and centimeter range because it is the amount of fluid and not the size of the microfluidic system which is important for practical use. In addition, such systems show significant differences from conventional fluidic systems because of the small amounts of fluid and often small system sizes. Miniaturization is accompanied by a change in the behavior of the fluid flow because surface-linked effects and electrostatic and electrokinetic forces dominate. New approaches are therefore necessary for the design, production and characterization of microfluidic components, e.g. micropumps and sensors. The constant energy density of the actuators results in their output falling, so that they are not comparable with conventional components in the macro sector. For this reason, external actuators are frequently employed and at times considerably increase the dimensions of the overall system. In addition, the physics and chemistry of the particles and molecules to be transported limit the miniaturization of microfluidic components.

Micro pumps have the task of metering very small amounts of liquid with at the same time low production costs and small external dimensions. The miniaturization of the pump makes use of physical effects which are only concomitant phenomena in macroscopic technology. As a consequence, the pumps can be divided into two groups, those with adapted macroscopic, and those with a new type of microscopic principle of action.

A large proportion of the pumps currently available or at the stage of laboratory model have microdisplacement pumps. The requirements for integration into a complete fluidic system are the main reason for this. Besides maximum capacity and maximum delivery pressure, important selection criteria include inter alia more cost-effective production, reproducible and stable delivery properties, simple filling and robustness in relation to perturbing influences. For reasons of clarity and relevance to this work, the following section deals only with the construction and mode of action of microdisplacement pumps. These consist substantially of three units, a pump chamber, an actuator for moving the fluid, and a valve unit to control the direction of flow. The pumping process is divided into two phases. In the suction phase, the actuator enlarges the chamber volume, a subatmospheric pressure is produced, and the fluid is sucked through the inlet. In the displacement phase, the actuator moves in the opposite direction and reduces the volume of the pump chamber. The fluid is pumped out of the pump through the outlet. The valve unit produces a directed liquid flow throughout the process.

The actuator principle and the construction of the valve unit are substantially determined by the required pump parameters, i.e. the pump output, the production process, the fluid properties, the energy supply and the permitted size. The two functional units are coordinated with one another and influence the operating properties of the pump.

Important parameters for comparing and selecting micropumps are the maximally achievable levels of delivery pressure and delivery rate.

Sensors transform physical, chemical or biological measured quantities into electrical measurement signals which are related to the measurements in an unambiguous way which is often, but not necessarily, linear. Microfluidic sensors are divided substantially into two groups. Flow sensors serve to detect the volumetric quantity or amount of substance passing the observed tube cross section per unit time. It is possible with the aid of an integrating unit to ascertain the total volume, which is important in particular for metering tasks. Chemical sensors by contrast detect the presence and the concentration of various substances, molecules or ions in the fluid, e.g. sensors to determine the pH. In relation to the aim of the present work, however, the following explanations are confined to flow sensors.

Flow sensors can be achieved with the aid of various physical laws which are utilizable even in macroscopic applications or only through the miniaturization. Depending on the measurement method, flow rates in the range from a few nanoliters up to some milliliters per minute can be measured.

In a thermally operated flow sensor, a temperature signal is fed by a heating element into the liquid flow and is detected again by a temperature sensor. The flow rate can be calculated therefrom on the basis of the measured signal expiry time and the distance covered.

Diabetes mellitus is a disorder in which the body is itself unable to produce and appropriately use any, or sufficient, amounts of insulin. Insulin is required to transport glucose from the blood into the cells of the body. The blood glucose level is continuously kept constant within narrow limits (60-100 mg % or 3.33-5.55 mmol/l). This takes place through the interplay of the two hormones insulin and glucagon.

Diabetes mellitus is diagnosed after taking blood by means of appropriate laboratory apparatuses. An elevated blood glucose level must be detected on at least two different occasions in order to confirm the diagnosis.

Diabetes mellitus is the term used when the glucose level measured in the blood plasma exceeds the stated value in at least one of the indicated cases:
a) fasting blood glucose—7.0 mmol/l or 126 mg/dl
b) blood glucose two hours after a dose of 75 mg of glucose (oral glucose tolerance test)—11.1 mmol/l or 200 mg/dl
c) blood glucose 11.1 mmol/l or 200 mg/dl associated with severe thirst (polydipsia), frequent urination (polyuria) or loss of weight.

Untreated diabetes leads to elevated blood glucose levels which may lead to various symptoms and late consequences such as, for example, polyneuropathy, microangiopathy, macroangiopathy, retinopathy, nephropathy and others. The risk of late damage from diabetes is less when the nonenzymatic glycation of erythrocytes (HbA1c level) is lower.

Diabetic coma is a life-threatening acute complication of diabetes. The blood glucose level may in such cases extend above 1000 mg/dl, associated with excessive acidity in the blood (metabolic acidosis). Diabetic coma can be induced inter alia by infections, intake of too much carbohydrate, alcohol abuse or incorrect insulin dosage.

A distinction is made between type 1 diabetes and type 2 diabetes. In type 1 diabetes there is an absolute insulin deficiency from the outset and treatment is possible only with insulin dosage.

Type 2 diabetes is characterized by a reduced insulin sensitivity and a relative insulin deficiency. Type 2 diabetes can usually be treated initially with dietetic measures and tablets. Insulin replacement frequently becomes necessary during the course of the disorder.

Type 2 diabetes has become a widespread disease predominantly in industrialized countries. Overeating, lack of exercise and obesity are regarded as the main cause. Type 2 diabetes can be effectively counteracted by exercise training and diabetic measures, especially aiming at weight reduction. It is also possible in the case of type 2 diabetes to employ oral antidiabetics such as, for example, acarbose, biguanides, sulfonylurea, glitazone and others. Therapy using insulin is necessary when the blood glucose level can no longer be kept in or near the normal range with sufficient permanence by means of said measures.

Various insulins are available for insulin therapy. A distinction is usually made according to the duration of action or chemical structure. An analog insulin has different amino acids at individual positions compared with human insulin. The properties may be changed thereby.

The rapid-acting insulins include human insulin and various rapid- and short-acting insulin analogs such as glulisin (proprietary name: Apidra), lispro (proprietary name: Humalog) and aspart (proprietary name: Novo Rapid).

Slow-acting or extended-acting insulins are NPH insulin (human insulin with an action extended by neutral protamine hagedorn), zinc insulins and various insulin analogs such as glargine (proprietary name: Lantus) and detemir (proprietary name: Levemir).

Also used in insulin therapy are mixed insulins and recently inhaled insulins.

Mixed insulins consist of a rapid-acting insulin and an extended-acting insulin in various mixing ratios. 10/90%, 25/75%, 30/70%, 50/50% mixtures are usual. Insulin therapy must always be accompanied by regular determinations of the blood glucose level.

In conventional insulin therapy, a defined amount of mixed insulin is injected at fixed times. More intensive conventional insulin therapy is employed predominantly for the therapy of type 1 diabetics. In this case, a basic supply is ensured with an extended-action insulin (basal) and a rapid-acting insulin (bolus) is given additionally at meal times.

Continuous subcutaneous infusion of insulin by means of a pump is suitable mainly for type 1 diabetics. The insulin is not injected but is passed into the body by a small pump. The pump is permanently present on the body. The insulin is supplied through a catheter with cannula. The insulin pump usually delivers rapid-acting insulin at small equal intervals over a prolonged period.

Glucagon-like peptide 1 (GLP1) is, alongside glucose-dependent insulinotropic peptide (GIP), one of the most important representatives of the incretins. Incretins are produced as hormones in the intestine and regulate inter alia the blood glucose level by stimulating insulin release in the pancreas.

The amount of intestinal hormones produced depends on the amount of carbohydrates taken in orally. The GLP1 level increases much more after oral glucose intake than after intravenous administration of glucose. It has been possible to show by investigations that intravenous infusion and subcutaneous injection of GLP1 in type 2 diabetics leads in many cases to complete normalization of the blood glucose level. A problem is that GLP1 is inhibited within a very short time by dipeptidylpeptidase IV (DPP-IV). Subcutaneous injection of GLP1 can maintain effective plasma concentrations over only about 1-2 hours. A solution in the direction of a persistent effect of GLP1 might be discoverable in the development of longer-acting GLP analogs or else inhibition of DPP-IV by pharmaceuticals.

Growth hormones are substances which stimulate growth in humans, animals and plants. Known examples are somatotropin (human), bovine somatotropin (cattle) and auxin and gibberellic acid (plant).

Somatotropin (STH) is also known under the names human growth hormone (HGH) and growth hormone (GH). STH is a peptide hormone with 191 amino acids. Production takes place in the anterior pituitary under the control of somatotropin-releasing factor (SRF; GHRH; GRF) from the hypothalamus. STH is absolutely necessary for normal linear growth. Reduced production or reduced response of the cells to STH results in short stature. Overproduction results in gigantism or acromegalie.

Short stature caused by growth hormone deficiency has been treated for some years by administration of STH. It was initially obtained from cadaver pituitaries before it became possible to produce STH by genetic manipulation in 1985.

Interferons are produced as tissue hormones by human or animal leukocytes, fibroblasts or T lymphocytes. An interferon is a protein or glycoprotein with an immunostimulating (e.g. antiviral) or antihormonal effect. Interferons are divided into alpha-interferons, beta-interferons and gamma-interferons. Interferons are obtainable from various manufacturers for indications such as viral diseases (e.g. SARS), cancer, multiple sclerosis, hepatitis B/C, hepatitis C.

A vaccine is a composition produced biologically or by genetic manipulation and comprising inter alia individual proteins and/or RNA or DNA fragments and/or killed or attenuated pathogens (e.g. influenza, SARS, pocks virus, pathogens of measles, mumps, rubella, poliomyelitis, pathogens of whooping cough).

Known types are live vaccines (e.g. cow pocks), attenuated live vaccines with attenuated viruses or bacteria (e.g. MMR vaccine, yellow fever, poliomyelitis) and dead vaccines with inactivated or killed viruses or bacteria or constituents thereof (e.g. influenza, cholera, bubonic plague, hepatitis A).

Heparins are substances employed therapeutically to inhibit blood coagulation. Heparins consist of in each case alternating sequences of D-glucosamine and D-glucuronic acid or L-iduronic acid. Chain length consisting of 5 units may be sufficient for anticoagulation.

The polysaccharide chains mostly have a molecular weight of between 4000 and 40,000. Besides unfractionated heparins, use is also made of low molecular weight fractionated heparins with a molecular weight of about 5000. Heparins are not absorbed from the gastrointestinal tract but must be administered parenterally. Heparins act by binding to antithrombin III and thus accelerating the inactivation of activated coagulation factors.

Lovenox (also known as clexane) is a commercially available pharmaceutical preparation with the pharmacologically active ingredient enoxaprin sodium. The active ingredient is one of the low molecular weight heparins with a linear dose-response relation and a constantly high bioavailability.

Areas of indication for Lovenox are the primary prophylaxis of deep vein thromboses, therapy of deep vein thromboses with or without pulmonary embolism, therapy of unstable angina pectoris and of the so-called non-Q-wave myocardial infarction, and thrombosis prophylaxis and anticoagulation during hemodialysis.

### EXAMPLE

The following statements describe the construction, mode of functioning and testing of a device according to the invention in the context of an automatic insulin pen based on selected examples.

The central component of the pen in this case is formed by the pump device which sucks the insulin out of the cartridge and injects it through the needle into the target tissue. This device comes into direct contact with the liquid. The insulin dosage is intended to take place with the aid of a sensor. This concept requires the use of cartridges and pen needles, so that the operating properties of the pump device must be adapted to these components. Important dimensioning parameters are the suction pressure and backpressure which can be generated with a constant delivery rate.

Firstly, the cartridge properties which are relevant in relation to the solution concept are ascertained. The core of the investigation is the necessary suction pressure to deliver the insulin, elicited by the friction between glass cylinder and stopper. Where possible, proposals for optimizing the pumping process in the pen to be developed are also to be made in this connection. Subsequently, the pressure drop on pumping the insulin to representative pen needles is to be ascertained. Based on these investigation results, a principle of action is to be selected for the pump device and then the suitability is to be demonstrated in a function test. The criteria are, besides the pump output, also the exchangeability, the compliance with medical requirements and the miniaturizability. Investigations of a sensor principle for detecting the insulin flow complete the section.

Cartridges and the injection needles form a coordinated functional unit for delivering insulin. Depending on the stopper speed and the pen needle diameter, when the stopper is advanced an equilibrium of forces is set up between the plunger stem force and the total of the forces resulting from the stopper friction and the hydrostatic pressure. The stopper position and the rate of advance are determined every time, neglecting the compressibility of the stopper, by the plunger stem position. The volume reduction in the cartridge in this case results in the superatmospheric pressure which pumps the liquid through the pen needle.

However, if the cartridges are used with the principle of action according to the invention there is a change in the variables in the system.

The following boundary conditions must be taken into account:

A subatmospheric pressure is produced in the cartridge when the liquid is sucked out. The subatmospheric pressure, also referred to as relative pressure, is theoretically limited to 101.03 kPa with normal external pressure. The maximally acting force of the stopper is limited to about 7.3 N by this pressure difference.

When the cartridge is free of air bubbles, the stopper speed depends exclusively on the stopper friction and the suction pressure. In the region of the starting friction, this may be small even if the suction pressure is high.

If the cartridge is free of air bubbles, the delivery rate depends on the stopper speed and varies accordingly.

The pressure drop at the pen needle has no influence on the processes in the cartridge with this principle of action.

The air dissolved in the liquid may be evolved as gas due to the subatmospheric pressure. Air bubbles present experience an increase in volume depending on the hydrostatic pressure. A very high suction pressure might lead to cavitation when the vapor pressure is reached. This must be absolutely avoided.

In the investigations on the suction process, both the hydrostatic pressure and the stopper position require measurement. They are intended to provide information about the suitability of the cartridges for this principle of action. Likewise, statements about the necessary suction capacity of the pump device and about optimization of the suction process are to be made. Particularly important aspects are the elastomeric stopper friction in the region of the starting friction and sliding friction. The investigations are to be carried out on a sufficiently large number and with cartridges of different batches in order to obtain informative results.

The test assembly consists of the four main components of syringe pump, pressure sensor, optical sensor and measurement computer with the LabView software (image 6.1). The syringe pump from TSE GmbH, model 540060 is connected by tubing and an injection needle to the cartridge and can be programmed and controlled with the aid of the computer. It is designed for suction and pressure operation and generates delivery rates within a fixed range. To determine the hydrostatic pressure, a pressure sensor supplied by Aktiv Sensor, model AUS±1.0 bar, likewise provided with tubing and injection needle, pierces the sealing disk of the cartridge. When correctly filled, the hydrostatic pressure can be measured relative to the air pressure in the cartridge, because no liquid flow takes place in the tubing and the capillary pressure in the injection needle is negligible. A linear sensor supplied by TAOS Inc., model TSLR1410R, is used to determine the stopper position. This sensor is arranged parallel to the cartridge and has a resolution of 400 dpi. On translumination with parallel light and shielding of the cartridge from ambient light, it detects the silhouette of the stopper. A data acquisition program calculates the stopper position from the silhouette with the aid of specific algorithms and interpolation with an accuracy up to 50 μm. The measurement is stored along with the hydrostatic pressure for further processing.

The experimental procedure follows a fixed pattern. Before starting the actual measurements, the syringe pump must be programmed with the desired pump sequence. The sequence may be composed of the removal of one or more doses with intermediate pauses or different delivery rates. A new cartridge is then to be put into the test assembly. The tubing filled with water and the syringe pump are then investigated for air bubbles and these are removed if necessary. Attention must be paid in general to minimizing the dead volume in the system. Finally, the fluid connections to the cartridge are to be made. The measurement can then be started. At the same time, the measurement computer actuates the syringe pump and starts to read the sensor signals. The measurement program converts the signals and stores them time-dependently in a file. To improve understanding and representation the hydrostatic pressure is always indicated relative to the air pressure with a negative sign. The absolute hydrostatic pressure of for example 60 kPa in the cartridge thus corresponds to a positive relative pressure of around 39 kPa in relation to normal air pressure.

Four batches each of 200 cartridges, one filled with distilled water and three with insulin Lantus Aspart (designation: L436, D029, D053) are available to investigate the medicament container. Variations in the filling of the various batches are to be identified in this way, and a reliable overall statement made possible.

A total of 44 test series were carried out during the measurements. The number of investigated cartridges was restricted, in view of the duration of the test, to 10 or 15 items per series of measurements.

At the start of the measurement, a short start-up time is followed by an increase in the suction pressure in the cartridge. Depending on the stopper friction, this reaches maximum values of up to 93 kPa. However, leaks in the system and evolution of gaseous atmospheric oxygen reduce a further rise in pressure even if the stopper is very firmly seated. Newly formed and previously present air bubbles expand. The force exerted by the external air pressure slowly sets the stopper in motion. After the starting friction has been overcome, the stopper experiences high acceleration and reaches a high speed in a short time. This is several times higher than in the quasi steady state of sliding friction. This is set up after the stopper movement has adapted to the delivery rate. The stopper speed is now constant. In the region of sliding friction there are observed to be pressure variations derived from changes in the frictional force between stopper and glass cylinder. After the pump is switched off, the pressure falls and the stopper ceases to move. In the phase of run-out friction, an equilibrium is formed between stopper and static frictional force.

1. A device for injecting a substance into the human or animal body comprising
a) at least one reservoir;
b) one or more outflow lines from a);
c) a pump mechanism which is attached between a reservoir from a) and an outflow line from b);
d) a component which is suitable for injection and which is functionally connected to an outflow line from b);
wherein the pump mechanism is driven by motive power.

2. The device as claimed in claim 1, wherein the pump mechanism comprises at least one pumping component and at least one further component which supplies the pumping component with drive energy.

3. The device as claimed in claim 1, wherein the pump mechanism comprises at least one pumping component and also has interfaces via which an externally present technical apparatus for generating motive power can be functionally connected to the pumping component, or via which a technical apparatus for generating motive power can be functionally coupled to the pumping component.

4. The device as claimed in claim 1, wherein the pump mechanism comprises a pumping component and a component which supplies the pumping component with drive energy as integrated unit.

5. The device as claimed in claim 2, wherein the pumping component consists of a tubing pump, diaphragm pump, gear pump or piezoelectrically operated pump.

6. The device as claimed in claim 3, wherein the pumping component consists of a tubing pump, diaphragm pump, gear pump or piezoelectrically operated pump.

7. The device as claimed in claim 4, wherein the pumping component consists of a tubing pump, diaphragm pump, gear pump or piezoelectrically operated pump.

8. The device as claimed in claim 1, wherein the pump mechanism is driven by a micromotor.

9. The device as claimed in claim 2, wherein a micromotor is used to generate the motive power to drive the pumping component.

10. The device as claimed in claim 3, wherein a micromotor is used to generate the motive power to drive the pumping component.

11. The device as claimed in claim 4, wherein a micromotor is used to generate the motive power to drive the pumping component.

12. The device as claimed in claim 1, wherein the energy source for the component generating the motive power consists of a battery, an accumulator, a solar cell or domestic current.

13. The device as claimed in claim 3, wherein the energy source for the component generating the motive power consists of a battery, an accumulator, a solar cell or domestic current.

14. The device as claimed in claim 1, wherein a transmission for stepping down or stepping up is inserted between the pump mechanism and the component for supplying the pump mechanism with motive power.

15. The device as claimed in claim 2, wherein a transmission for stepping down or stepping up is inserted between the pumping component and the component for supplying the pumping component with motive power.

16. The device as claimed in claim 3, wherein a transmission for stepping down or stepping up is inserted between the pumping component and the component for supplying the pumping component with motive power.

17. The device as claimed in claim 4, wherein a transmission for stepping down or stepping up is inserted between the pumping component and the component for supplying the pumping component with motive power.

18. The device as claimed in claim 1, wherein the reservoir has an inflexible outer wall.

19. The device as claimed in claim 1, wherein the reservoir has a flexible outer wall.

20. The device as claimed in claim 1, wherein the reservoir consists of a commercially available cartridge for receiving a medicament.

21. The device as claimed in claim 1, wherein the component suitable consists of a cannula.

22. The device as claimed in claim 2, further comprising at least one electronic component for checking, monitoring and/or controlling the pumping component and/or the component which supplies the pumping component with motive power.

23. The device as claimed in claim 3, further comprising at least one electronic component for checking, monitoring and/or controlling the pumping component and/or the component which supplies the pumping component with motive power.

24. The device as claimed in claim 4, further comprising at least one electronic component for checking, monitoring and/or controlling the pumping component and/or the component which supplies the pumping component with motive power.

25. The device as claimed in claim 1, further comprising a flow sensor to determine the amount of the substance which is removed from the reservoir, and the amount which is used for injection.

26. The device as claimed in claim 1, further comprising a flow sensor to determine the amount of the substance which is removed from the reservoir, or the amount which is used for injection.

27. The production of a device as claimed in claim 1 where
a) a component to receive a reservoir is provided;
b) a reservoir is provided;
c) an outflow line for removing a substance from the reservoir is provided;
d) a pump mechanism is provided;
e) a component for injecting a substance is provided;
f) a flow sensor is provided;
g) electronic components for storage and/or data processing and/or data transfer are provided;
h) the individual constituents as described in a) to g) are joined together to give a functional unit.

28. A medical device suitable for administering a substance into the human or animal body, avoiding the gastrointestinal tract, comprising a device according to claim 1.

29. A medical device according to claim 28, wherein the substance is a pharmaceutical.

30. A medical device according to claim 29, wherein the pharmaceutical is an insulin.

31. A medical apparatus for injecting a pharmaceutical into the human or animal body, comprising
a) a base element for mounting at least one further component;
b) a component for removing air bubbles from the liquid intended for injection;
c) a component for presetting the amount of liquid intended for injection;
d) a component for displaying the amount of liquid intended for injection;
e) a component for initiating the injection of liquid; and
f) a component consisting of a device as claimed in claim 1.

32. The medical apparatus as claimed in claim 31, which comprises at least one means for storing and processing data and signals.

33. The medical apparatus as claimed in claim 31, which comprises at least one means for storing or processing data or signals.

34. The medical apparatus as claimed in claim 31, which comprises at least one interface for transmitting data or signals to or from an external technical unit which is configured for the storage or processing of data or signals.

35. The medical apparatus as claimed in claim 34, wherein the external technical unit consists of a PC.

36. The medical apparatus as claimed in claim 31, wherein the pharmaceutical intended for injection consists of insulin.

37. The medical apparatus as claimed in claim 36, in which the insulin is a long-active or a short-active insulin.

38. The medical apparatus as claimed in claim 31, wherein the substance intended for injection consists of GLP-1.

39. The medical apparatus as claimed in claim 31, wherein the substance intended for injection consists of Lovenox.

40. The production of a medical apparatus as claimed in claim 31, where
a) a base element for mounting at least one further component is provided;
b) a component for removing air bubbles from the liquid intended for injection is provided;
c) a component for presetting the amount of the liquid intended for injection is provided;
d) a component in the form of a display is provided;
e) a component in the form of a release mechanism is provided;
f) a device as claimed in claim 1 is provided; and
g) the individual constituents as described in a) to f) are combined to give a functional unit.

41. A medical apparatus for the administration of a substance, whose pharmaceutical activity is diminished or lost in the gastrointestinal tract, for the prophylaxis or therapy of a disease or dysfunction of the body comprising a medical apparatus according to claim 31.

42. An apparatus according to claim 41, wherein the disease is diabetes.

43. A medical apparatus according to claim 41, wherein the substance is insulin.

44. A medical apparatus according to claim 41, wherein the substance is GLP-1.

45. A medical apparatus according to claim 41, wherein the substance is an interferon.

46. A medical apparatus according to claim 41, wherein the substance is growth hormone.

47. A medical apparatus according to claim 41, wherein the substance is heparin.

48. A medical apparatus according to claim 41, wherein the substance is Lovenox.

49. A medical apparatus according to claim 41, wherein the substance is a vaccine.
